(19) United States
(12) Patent Application Publication
(10) Pub. No.: US 2006/0249155 A1
(43) Pub. Date: Nov. 9, 2006

(54) Portable non-invasive ventilator with sensor

(75) Inventor: Anthony Gambone, Silver Springs, MD (US)

Correspondence Address: ROBERT D. FISH;RUTAN & TUCKER LLP, 611 ANTON BLVD 14TH FLOOR, COSTA MESA, CA, 92626-1931, US

(73) Assignee: China Resource Group, Inc.

(21) Appl. No.: 11/416,517

(22) Filed: May 2, 2006

Related U.S. Application Data
(60) Provisional application No. 60/677,529, filed on May 3, 2005.

(51) Int. Cl. A61M 16/00; A62B 7/04

(52) U.S. Cl. 128/204.23; 128/204.18; 128/204.21; 128/204.26

(57) ABSTRACT

A ventilator has a microprocessor that assists in automatically determining an appropriate breathing mode, and either the microprocessor or some other circuitry utilizes the mask to deliver that mode. Contemplated breathing modes include all of the recognized breathing modes (i.e, controlled mandatory ventilation, synchronized intermittent mandatory ventilation, pressure controlled and/or pressure supported ventilation, and high frequency ventilation) as well as other breathing modes.

This application claims priority to U.S. provisional application Ser. No. 60/677529 filed May 3, 2005.

### FIELD OF THE INVENTION

The field of the invention is breathing assistance devices. CL BACKGROUND P Numerous non-invasive ventilation devices have been developed over the years. Perhaps the simplest is a simple mask with compressible balloon bag commonly used by emergency medical personnel. A major problem with such devices, however, is that they need to be employed by skilled personnel in order to be effective.

A few devices have been suggested that would provide a more automatic solution, and could therefore be more readily used by non-professionals. A recent example is that described in RE 38533. This, and all other referenced patents and applications are incorporated herein in their entirety. Unfortunately, the devices contemplated by RE 38533 is not fully automatic in that the user must determine the nature of the distress condition for himself and then select the appropriate breathing mode.

Another device intended improve usability among non-professionals is that set forth in U.S. Pat. No. 6,289,890. That patent, however, merely describes standard ventilators equipped with voice prompts. While being potentially advantageous, the voice prompts still do not automatically determine the nature of the distress condition and then automatically apply the appropriate breathing mode.

Thus, there is still a need for a more automatic breathing assistance device.

### SUMMARY OF THE INVENTION

The present invention provides systems and methods in which a microprocessor assists in automatically determining an appropriate breathing mode, and either the microprocessor or some other circuitry utilizes the mask to deliver that mode.

### BRIEF DESCRIPTION OF THE DRAWING

FIG. 1 is a schematic of a ventilation system according to the inventive subject matter.

### DETAILED DESCRIPTION

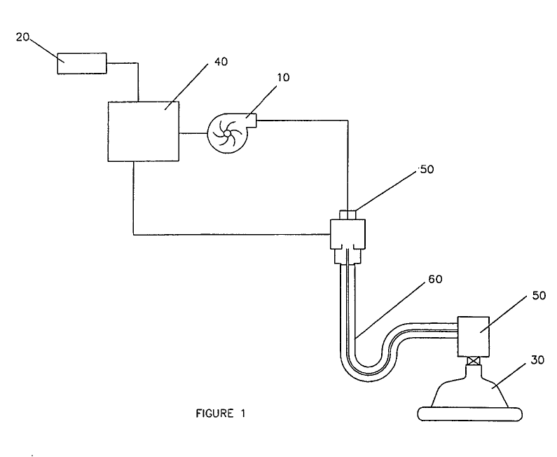

In FIG. 1 a ventilation system 1 generally includes a pressurized gas source 10; a portable power source 20; a face mask 30 coupled to the pressurized gas source 10; a microprocessor 40; a sensor 50 that provides information to the microprocessor to assist in automatically determining an appropriate breathing mode; and a circuitry 60 that utilizes the mask to deliver that mode.

The pressurized gas source 10 can be any of the myriad types known in the art, or later developed. Specifically contemplated is a limited drag turbine flow generator such as that described in U.S. Pat. No. 6,526,970. The gas source 10 shown in the figure pressurized atmospheric air, but could additionally or alternatively include a supply of gas having a higher content of oxygen than the ambient air.

The power source 20 can be any suitable power source, including for example a batter, super capacitor, hand carryable hydrogen fuel cell, and the like.

The face mask 30 can be any suitable face mask. Preferred face masks cover both the nose and mouth of the victim, and it is contemplated that a given system may include multiple sized masks to accommodate victims of different sizes.

The microprocessor 40 can be any suitable microprocessor, including for example general purpose microprocessors such as Pentium chips, as well as special purpose microprocessors. The microprocessor 40 would, of course, be coupled with needed memory, power supply, I/O devices, control circuitry and so forth.

Sensor 50 can sense patency of an airway of a victim, which in a preferred embodiment is a sound transducer. A freestanding sound transducer that provides an output signal characteristic of the respiratory tract is described in U.S. Pat. No. 5,666,960, and can readily be implemented in the system.

Additional or alternative sensors can include a pressure-drop pneumotach known in the art. Such as device can measure air flow, pressure, breathing depth, rate of breathing, and other time-related physiology.

Still other sensors can additionally or alternatively include an infra-red end-tidal CO₂ device, a high speed polaragraphic oxygen analyzer, or other chemical composition analyzers.

Contemplated breathing modes include all of the recognized breathing modes (i.e, controlled mandatory ventilation, synchronized intermittent mandatory ventilation, pressure controlled and/or pressure supported ventilation, and high frequency ventilation) as well as other breathing modes. An important consideration here is that the software executed by the microprocessor determines an appropriate breathing mode. Ideally, the microprocessor would determine an optimal breathing mode.

As used herein, the term “automatically determining an appropriate breathing mode” should be interpreted broadly to include sufficient analysis to select an appropriate breathing breath delivery, even if the software does not specifically identify the mode by name or otherwise.

It is contemplated that the ventilation systems described herein could be integrally included in a combined system that includes an automatic external defibrillator. Such a combined system could use common microprocessor, power supply, display, voice or other interface, controls, and so forth.

Systems can also include emergency personnel contact facilities, such as 911 calling, GPS (global positioning system) receiver, and so forth.

It should be apparent, however, to those skilled in the art that many more modifications besides those already described are possible without departing from the inventive concepts herein. Moreover, in interpreting the disclosure, all terms should be interpreted in the broadest possible manner consistent with the context. In particular, the terms “comprises” and “comprising” should be interpreted as referring to elements, components, or steps in a non-exclusive manner, indicating that the referenced elements, components, or steps could be present, or utilized, or combined with other elements, components, or steps that are not expressly referenced.

What is claimed is:

1. A ventilator system comprising:
a pressurized gas source having a portable power source;
a face mask coupled to the pressurized gas source;
a microprocessor;
a sensor that provides information to the microprocessor to assist in automatically determining an appropriate breathing mode;
a circuitry that utilizes the mask to deliver that mode.

2. The system of claim 1 wherein the pressurized gas source comprises a turbine.

3. The system of claim 1 wherein the sensor senses patency of an airway of a victim.

4. The system of claim 3 wherein the sensor comprises a sound transducer.

5. The system of claim 1 wherein the sensor senses a rate of breathing.

6. The system of claim 1 wherein the sensor senses a depth of breathing.

7. The system of claim 1 wherein the sensor senses a composition of a component of exhaled breath.

8. The system of claim 1 wherein the sensor senses breath flow velocity.

9. The system of claim 1 wherein the microprocessor selects the mode from a plurality of modes.

10. The system of claim 1 wherein the plurality of modes includes at least one of controlled mandatory ventilation, synchronized intermittent mandatory ventilation, pressure controlled and/or pressure supported ventilation, and high frequency ventilation.
